(19) United States
(12) Patent Application Publication
(10) Pub. No.: US 2007/0092876 A1
(43) Pub. Date: Apr. 26, 2007

(54) Method and system for cell and/or nucleic acid molecules isolation

(76) Inventor: Guolin Xu, Singapore (SG)

Correspondence Address: OSTROLENK FABER GERB & SOFFEN, 1180 AVENUE OF THE AMERICAS, NEW YORK, NY, 100368403, US

(21) Appl. No.: 10/535,334

(22) PCT Filed: Nov. 10, 2003
(86) PCT No.: PCT/SG03/00261

§ 371 Date: Aug. 21, 2006

Related U.S. Application Data
(60) Provisional application No. 60/427,148, filed on Nov. 18, 2002.

(51) Int. Cl. C12Q 1/68; C12N 1/08

(52) U.S. Cl. 435/6; 435/270

(57) ABSTRACT

The present invention relates to methods and system for tissue cell and/or nucleic acid molecule isolation. In particular, to a method for isolating nucleic acid molecules from tissue samples comprising: i) treating a tissue sample with at least one enzyme for tissue dissociation; ii) adding a lytic solution; and iii) isolating nucleic acid molecules. The method further comprises a step of applying hydrodynamic shear force to the product of step (i). The methods and/or system according to the invention are adaptable for use with micromechanical and/or automated processes.

### FIELD OF THE INVENTION

The present invention relates to methods and systems for cell and/or nucleic acid molecules isolation. In particular, the methods and/or systems according to the invention are adaptable for use with micromechanical and/or automated processes.

### BACKGROUND OF THE INVENTION

Analysis of the nucleic acids in tissues is performed for many purposes, including forensic sciences, the study of diseases medical sciences pharmacological drug discovery and development and clinical diagnostics. This study of the nucleic acids typically requires extracting the nucleic acids from the tissue. A step in nucleic acid extraction is tissue homogenization.

A tissue usually contains many cells that are joined together by a biological matrix that provides mechanical strength to the tissue. The tissue homogenization step breaks up the biological matrix. The biological matrix is typically rich in collagen, often as much as 90% collagen.

After the homogenization step, the cells must also be broken up in a cell disruption step so that the nucleic acids they contain may be analyzed. The homogenization and cell disruption steps are typically accomplished simultaneously or by the homogenization step breaking up some of the cells first followed by the cell disruption step, which completes the cell disruption process. FIG. 1 provides a flow chart of nucleic acid extraction and analysis, see also Huang et al., 2002, Anal. Bioanal. Chem., 372, 49-65.

The tissue homogenization step conventionally involves using mechanical force to disrupt the tissue, and the cell disruption step conventionally involves using chemicals or enzymes. For disrupting biological samples such as fresh and frozen mammal tissues, or culture cells, conventional mechanical methods are used. These methods include: 1) using a motorised mechanical homogeniser that employs a component like a blender to generate shear force to physically break up solid tissues and release all intracellular components into the surrounding medium; 2) using a high-pressure homogeniser that employs impingement of high liquid shear force in orifice to explode the cells; 3) using a bead mill that breaks up cells by shear force generated due to grinding and collisions between beads; and 4) using a sonicator that employs ultrasonic waves to generate intense pressure waves with enough energy to break cell membranes.

The mechanical tissue homogenization breaks up the tissue so that the chemicals or enzymes can penetrate the sample and the cells in the tissue. Without tissue homogenization, the chemicals or enzymes in the cell disruption step would only affect some of the cells in the tissue sample. Tissue homogenization breaks up some of the cells, but the chemical and enzymatic treatments are needed to disrupt all the cells and to help separate the nucleic acids from the rest of the cell. Other complex tasks to complete the analysis are performed after the nucleic acids have been extracted, including amplification and detection of the nucleic acids.

The task of preparing nucleic acids for analysis has conventionally been a time-consuming and labor-intensive process. These methods have several drawbacks. One of these drawbacks is that the mechanical homogenization process does not allow a full dissociation of the tissues, as cells may still be clustered together. A further problem is that the during the mechanical tissue homogenization step, some cells of the tissue sample may be broken so that RNAases are released from the cells. RNAses are ribonucleasess that destroy RNA polynucleic acids so that nucleic acid analysis becomes ineffective. Another further problem is that the homogenization process for the preparation of cell lysate from tissue is performed manually with an electric homogeniser, one sample at a time, resulting in the need for frequent washes of the homogeniser tip to prevent cross contaminations. Other further problems are that: i) a large tissue size is required due to the large working volume of these devices; ii) these devices are complex in structure and bulky in size so they are not easy to implement inside microfluidic devices; iii) they are very difficult to automate; iv) they are easily amenable to operation error and cross-contamination; v) some of these methods generate a considerable amount of heat that degrade the quality of the intracellular components of interest; and vi) most of them are not powerful enough to disrupt fresh or frozen solid tissues.

Recent advances in μ-fluidics and microelectromechanical systems (MEMS), Micro Total Analytical Systems (μTAS) and biochip technology have led to the miniaturisation of many micro-scale analytical instruments. The advantages of miniaturisation in fluid processing include improved efficiency with regard to sample size, response times, cost, analytical performance, process control, integration, throughput and automation (de Mello, Anal. Bioanal. Chem. 372:12-13, 2002).

The homogenization and the cell disruption steps of the process, however, continue to be performed in a time-consuming and labor-intensive manner. Indeed, it has been difficult to automate, make robots, or make micromechanical devices that perform homogenization and cell disruption due to the miniaturized nature of systems like MEMS and μTAS.

### SUMMARY OF THE INVENTION

The present invention addresses the problems above and provides new methods and/or systems for cell isolation and/or for nucleic acid molecules isolation. In particular, the methods and systems according to the invention are adaptable for use with micromechanical and/or automated processes. The method and/or systems of the invention do not require mechanical homogenization step so that automatic, robotic, or micromechanical approaches to tissue dissociation may be accomplished.

According to one aspect, the invention provides a method for isolating nucleic acid molecules from tissue samples comprising:
i) treating a tissue sample with at least one enzyme for tissue dissociation;
ii) adding a lytic solution;
iii) isolating nucleic acid molecules and/or proteins.

The method and system of the invention relate to tissue sample dissociation using at least one enzyme for the tissue dissociation. Accordingly, the method and system of the invention do not require a mechanical homogenization step.

In particular, the method of the invention further comprises a step of applying hydrodynamic shear force to the product of step (i).

The present method and system therefore utilize hydrodynamic shear force to break up the tissue sample so that the tissue is efficiently disrupted and cells can be released from the tissue sample. Further, the applied hydrodynamic shear force breaks up the tissue sample so that it becomes small enough to pass through devices, like miniaturised and/or microfluidic devices.

The enzyme for tissue dissociation may be conveniently chosen according to the tissue sample desired to be dissociated. The tissue sample may be animal-, human-, or agricultural-originated tissue. In particular, the enzyme for tissue dissociation may be a protease, cellulase, lipase, and the like. For example, any of the following protease or a mixture thereof may be used: collagenase, trypsin, chymotripsin, elastase, papain, chymopapain, hyaluronidase, pronase, dispase, thermolysin, bromelain, cathespines, or pepsin, or a mixture thereof.

The released cells are treated with a lytic solution. The cell membrane is broken to release intracellular components, in particular nucleic acids and/or proteins. Nucleic acid molecules may be isolated and recovered according to any standard technique known in the art. For example, nucleic acid molecules may be isolated by adding beads coated with at least one linker and thus recovering the nucleic acid molecules bound to the linkers.

The isolated nucleic acid molecule is mRNA, RNA and/or DNA.

According to a further aspect, the invention also provides a method for cell isolation from tissue samples comprising:
(a) treating a tissue sample with at least one enzyme for tissue dissociation;
(b) applying hydrodynamic shear force to the product of step (a);
(c) recovering the isolated cells.

The recovered isolated cells may be preserved or stored for future use or may be used to extract nucleic acid molecules as mentioned above.

According to another aspect, the invention provides a system (device) for isolation of cells from tissue samples, the system comprises an enzymolytic tissue dissociation chamber and a tissue disruption channel.

According to a further aspect, the invention provides a system (device) for isolation of nucleic acid molecules from tissue samples, the system comprises an enzymolytic tissue dissociation chamber and a tissue disruption channel The tissue disruption channel is advantageous in that it allows the hydrodynamic shear force to break up the tissue sample so that it becomes small enough to pass through the channel.

In particular, the tissue disruption channel in the system comprises:
an inlet port;
at least one region of constriction; and
an outlet port.

The tissue disruption channel at the region(s) of constriction has a smaller cross-sectional area compared to the overall cross-sectional area of the disruption channel. The region(s) of constriction help to gradually reduce the size of the tissue sample until it is efficiently disrupted.

The enzymolytic tissue dissociation chamber may be of a small size. Chambers of small sizes are adaptable for use with micromechanical and/or automated processes. The chambers, for example, may have a volume of less than 100 μl, less than 50 μl, less than 10 μl, or less than 5 μl.

The proteolytic tissue dissociation chamber may be operably connected to at least one other chamber of the system. For example, the other chamber(s) is used for holding at least one protease, holding buffers, holding protease inhibitors, holding stains or visualization agents, or serving as receptacles for waste products or nucleic acid molecules.

In particular, the system of the invention may be a biological microelectromechanical system (bioMEMS) and/or a fully automated complete micrototal analytical system (μTAS). It may also be an automated nucleic acid and/or protein extractor.

In particular, the system of the invention is a system for isolation of cells from tissue samples, comprising:
a first chamber for incubation of a mixture of: at least one tissue sample, at least one enzyme for dissociation of the tissue sample, and buffer solution;
a second chamber which is as a tissue disruption channel for generating hydrodynamic shear force;
and optionally a chamber for cells collection, and
a chamber for waste collection;
and optionally the chambers are connected to each other.

The system of the invention also provides a system for isolation of nucleic acid molecules from tissue samples, comprising:
a first chamber for incubation of a mixture of: at least one tissue sample, at least one enzyme for dissociation of the tissue sample, and buffer solution;
a second chamber which is as a tissue disruption channel for generating hydrodynamic shear force;
a third chamber including a lytic solution;
a fourth chamber for the collection and isolation of nucleic acid molecules and/or proteins; and
a fifth chamber for waste collection;
wherein optionally the chambers are connected to each other.

Any one of the system (device) of the invention optionally comprise a port for input tissue sample, and inlet and outlet of the tissue disruption channel for connecting fluids and pump, respectively.

The tissue disruption channel comprises the disruption components as described above.

The system may comprise a chamber containing beads, matrixes and/or carriers for the isolation of nucleic acid molecules. In particular, beads coated with at (east one linker for isolation of nucleic acid molecules may be used. The beads may be magnetic beads.

Further, the system may be part of a diagnostic integrated system, which is suitable for forensic testing, clinical diagnostics, veterinary, agricultural diagnostics, and like.

According to another aspect, the invention provides a method for isolating cells from a tissue sample, the method comprising:
incubating in a first chamber a mixture of: at least one tissue sample, at least one enzyme for dissociation of the tissue sample, and buffer solution;
disrupting the tissue sample in a second chamber which is a tissue disruption channel for generating hydrodynamic shear force;
recovering the cells, optionally, in a third chamber; and
optionally recovering the waste in a fourth chamber.

According to a further aspect, the invention provides a method for isolating nucleic acid molecules from a tissue sample, the method comprising:
incubating in a first chamber a mixture of: at least one tissue sample, at least one enzyme for dissociation of the tissue sample, and buffer solution;
disrupting the tissue sample in a second chamber which is a tissue disruption channel for generating hydrodynamic shear force;
lysing cells isolated from the tissue disruption channel in a third chamber; and
collecting and isolating desired nucleic acid molecules in a fourth chamber.

The incubation in the first chamber may be carried out at constant temperature.

The method comprises applying hydrodynamic shear force within the tissue disruption channel to gradually reduce the tissue sample size until it is fully disrupted and cells are released.

The collection of the nucleic acid molecules may be collected and/or isolated according to any standard method known in the art. For example, nucleic acid molecules may be collected from the solution by: adding beads coated with at least one linker and thus the nucleic acid molecules bound to the linkers are recovered.

According to a particular aspect, the method according to the invention comprises providing a tissue sample of less than about 10 mm³ or less than about 3 mm³ and exposing the tissue sample to at least one enzyme for dissociation and optionally applying hydrodynamic shear force until the tissue is efficiently disrupted.

### BRIEF DESCRIPTION OF THE FIGURES

FIG. 1 is a flow chart of a conventional scheme for nucleic acid analysis.

FIG. 2 depicts an agarose gel showing that some embodiments of the invention (lanes 3-6) are as effective as conventional methods (lane 7).

FIG. 3A is a plan view of a microfluidic tissue digester incorporating a proteolytic tissue dissociation chamber.

FIG. 3B is a perspective view of the device of FIG. 3A.

FIG. 4 shows the agarose gel of the total RNA from fresh tissue by using the invented dissociation method. It shows that RNA isolated is not degraded. Table 5 shows the total RNA yield comparison. Data show the total RNA yield variation is small. The invented dissociation method is reliable. Lane M:Marker, Lane 1-4: RNA isolated by trypsin digestion, Lane 5-6: RNA isolated by homogenizer.

FIG. 5: Agarose gel of total RNA from frozen tissues (Lane 1-4).

FIG. 6 shows the agarose gel of mRNA extracted from the invented tissue dissociation method. The genes that we want to synthesize are shown in the figure. mRNA is intact by using the dissociation method invented. Full-length cDNAs are synthesized by SuperScript (Invitrogen). M: marker, lane 1: βactin, lane 2: β-microglobulin, lane 3: cyclophilin, lane 4: TP53, and lane 5: c-myc.

FIG. 7 shows the agarose gel of mRNA extracted from human breast tissue dissociated by the invented method. The genes that we want to synthesize are shown in the figure. Full-length genes are from frozen human breast tissue by SuperScript (Invitrogen). Lane 1: 100 bp DNA ladder, Lane 2:

GAPDH, Lane 3: β-actin, Lane 4: CD59, Lane 5: keratin 19, Lane 6: TP53, Lane 7: Histone H4, Lane 8: Maspin, Lane 9: α-1-antichymotrypsin.

FIG. 8: Microfluidic tissue disruption device, 1: tissue input/incubation chamber, 2: disruption channel, 3: inlet for fluid, 4: outlet for fluid.

FIG. 9: Detailed drawing of the tissue disruption components, 5: inlet port, 6: region of constriction, 7: outlet port.

FIG. 10: Some possible designs of disruption components.

FIG. 11(A, B): FIG. A shows a section of a sandwich structure of a microfluidic device made of stainless steel comprising: polycabonate upper and lower layers,: and acrylic tape bonding layer,and a stainless steel layer. In this structure, the stainless steel features layer is bonded with the upper and lower layer to form the disruption channels. FIG. B shows a section of the structure of a microfluidic device made of polycarbonate using hot embossing or CNC, and bonded by heat diffusion.

FIG. 12: A biomolecular extraction and purification device, 8: water reservoir, 9: lysis buffer, 10: magnetic beads, 11: washing buffer A, 12: washing buffer B, 13: elute buffer, 14: product reservoir, 15: valve unit, 16: reagent channel, 17: disruption/mixing channel, 18 & 19: connected to pump, 20: tissue inlet/incubation chamber.

FIG. 13: Cell yield comparison between the bench-top conventional method and MEMS-based device.

FIG. 14: Agarose gel of β-actin RT_PCR synthesis. Lane from M: Marker, lane 1: β-actin from microfluidic device sample, lane 2: β-actin from motorised homogeniser sample.

FIG. 15: Agarose gel of TP53 and Cyclophilin RT-PCR Synthesis, M: marker, lane 1: TP53 from microfluidic device sample, lane 2: TP53 from motorised homogeniser sample, lane 3: cyclophilin from microfluidic device sample, and lane 4: cyclophilin from motorised homogenizer sample.

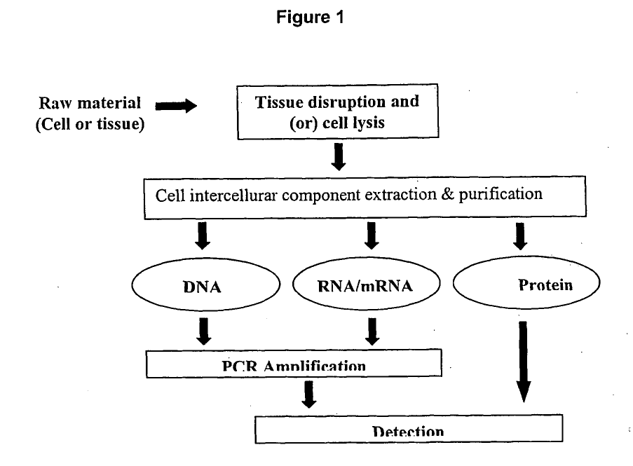

### DETAILED DESCRIPTION OF THE INVENTION

The invention provides methods and systems for processing tissue samples for nucleic acid molecules extraction and isolation that may be adaptable for use with micromechanical devices and/or automated processes. An embodiment of the invention is a method for performing tissue dissociation without a step of mechanical homogenization of the tissue. The tissue is dissociated using at least one enzyme for dissociation. For example, at least one protease (for example, trypsin or collagenase), cellulase or lipase, or a mixture thereof, can be applied as a solution that contacts the tissue and dissociates it.

The process of adding at least one enzyme to a tissue sample for dissociation can be performed quickly and requires no complex equipment. This is advantageous because the tissue dissociation process may thereby be automated and may be incorporated into micromechanical devices. Micromechanical devices include biological microelectromechanical systems (bioMEMS) and fully automated complete micro total analytical systems (μTAS).

Examples of conventional mechanical tissue homogenization methods are listed in Table 1.

TABLE 1
Conventional mechanical tissue and cell homogenization

| Cell disruption | | General |
|---|---|---|
| method | Application | procedure |
| Sonication: | Cell | Sonicate cell |
| Ultrasonic waves | suspensions | suspension in |
| generated by a | | short bursts to |
| sonicator lyse | | avoid heating. |
| cells through | | Cool on ice |
| shear forces. | | between bursts. |
| Complete shear- | | |
| ing is obtained | | |
| when maximal | | |
| agitation is | | |
| achieved, but | | |
| care must be | | |
| taken to min- | | |
| imize heating | | |
| and foaming. | | |
| French pressure | Microorganisms | Place cell |
| cell: | with cell walls | suspension |
| Cells are lysed | (bacteria, algae, | in chilled |
| by shear forces | yeasts) | French pressure |
| resulting from | | cell. Apply |
| forcing cell | | pressure and |
| suspension | | collect extruded |
| through a small | | lysate. |
| orifice under | | |
| high pressure. | | |
| Grinding: | Solid tissues, | Tissue or cells |
| Some cell types | microorganisms | are normally |
| can be opened | | frozen with |
| by hand grinding | | liquid nitrogen |
| with a mortar | | and ground to |
| and pestle. | | a fine powder. |
| | | Alumina or |
| | | sand may aid |
| | | grinding. |
| Mechanical | Solid tissues | Chop tissue into |
| homogenization: | | small pieces if |
| Many different | | necessary. Add |
| devices can be | | chilled homog- |
| used to mechan- | | enization buffer |
| ically homogenize | | (3-5 volumes |
| tissues. Hand- | | to colume of |
| held devices such | | tissue). Homogenize |
| as Dounce or | | briefly. Clarify |
| Potter-Elvehjem | | lysate by |
| homogenizers can | | filtration and/or |
| be used to | | centrifugation. |
| disrupt cell | | |
| suspensions | | |
| or relatively | | |
| soft tissues. | | |
| Blenders or | | |
| other motorized | | |
| devices can be | | |
| used for larger | | |
| samples. Homog- | | |
| enization is | | |
| rapid and poses | | |
| little danger | | |
| to proteins | | |
| except by the | | |
| proteases that | | |
| may be | | |
| liberated upon | | |
| disruption. | | |
| Glass bead | Cell | Suspend cells in |
| homogenization: | suspensions, | an equal volume |
| The abrasive | microorganisms | of chilled lysis |
| actions of the | | solution and |
| vortexed beads | | place into a |
| break cell walls, | | sturdy tube. Add |
| liberating the | | 1-3 grams of |
| cellular contents. | | chilled glass |
| | | beads per gram |
| | | of wet cells. |
| | | Vortex 1 |
| | | minute and |
| | | incubate |
| | | cells on ice |
| | | 1 minute. |
| | | Repeat vortexing |
| | | and chilling two |
| | | to four times. |

Further, the dissociation process may be performed so that the cells in the tissue sample are substantially not disrupted until the cell disruption (lysis) step. After dissociation of the tissue, the cells of interest may be separated from the rest of the tissue so that the contents of a desired subset of cells may be probed instead of all the cells in the tissue. Screening and/or separation of the cells of interest may be carried out according to standard methodologies.

Further, since the cells may be kept intact through the tissue dissociation step, the RNAases in the cells are kept essentially within the lysosomes in the cells and are thereby sequestered within the cell. RNAses are ribonucleases that destroy RNA polynucleic acids so that nucleic acid analysis becomes ineffective. RNAses are conventionally inhibited using RNAase inhibitors. Since RNAases may essentially be sequestered with the cells using any embodiment of the invention, the need for RNAase inhibitors, and the need for vigilance in their administration, may be eliminated. The intact cells need not necessarily be viable. Intact refers to a state of the membrane of the cells, including the cellular wall and lysosomes. Viability refers to the ability to remain alive. Cells may thus be intact but unviable.

Avoiding the effects of RNAases is important. It is well known that RNAs are fragile and rapidly degradable by RNAses present in a tissue sample as well as contaminations from human sweat, including that present on fingertips. Other than ensuring that all instruments, containers, and working areas are RNAase-free, the technicians must be careful not to allow freshly harvested samples to remain at room temperature unpreserved, frozen samples to defrost, or mechanical tissue disruption to take place without the presence of nuclease inhibitors. Certain embodiments of the invention remove all these meddlesome technicalities. For example, a chamber of the bioMEMs can receive the sample immediately after biopsy or tissue harvest, potentially removing the need for preservation procedures. Further, a fully automated sample preparation requires no human interference greatly minimising contaminative nucleases found in human sweat.

Some conventional methods for isolating cells use proteases to treat a tissue. A protease is an enzyme that cleaves or catalyzes the cleavage of peptidic chemical bonds. A peptidic chemical bond is a chemical bond that joins two or more amino acids, for example: a bond formed between two amino acids of a protein. For example, Dwulet et al., in U.S. Pat. No. 5,952,215 and Uchida, in U.S. Pat. No. 6,238,922, describe exposing tissue to the protease collagen and Freshney describes exposing tissue to trypsin, see RI Freshney, Freshney's Culture of Animal Cells, Chapter 11: Primary Culture (1999). Such methods are not, however, directed to the isolation of nucleic acids. Instead, they are directed to degrading the structure of a tissue to allow cells to be isolated and cultured, a very different goal unrelated to nucleic acid isolation. Consequently, such methods are inoperable to achieve the embodiments of the invention because those methods are directed to optimizing cell viability, do not thoroughly break the bonds in the tissue, do not homogenize the tissue, and conventionally use different temperatures, concentrations, and/or durations of proteolytic exposure.

MEMS are conventionally useful only with cellular samples e.g., blood cells and microorganisms. A further advantage of certain embodiments of the invention, however, is that micromechanical devices may now be adapted to be used with solid tissues using the present invention. Table 1 refers to conventional mechanical homogenization methods. A review of these methods shows that they use processes that are difficult to automate or adapt to a micromechanical device. For example, sonication of tissue tends to cause heating and foaming, while grinders and glass beads are difficult to reduce in size. Although many μ-fluidic modules have been demonstrated in the past decade to perform basic nucleic acid extraction and purification processes, the sample preparation step is conventionally left off chip. The reason is that the sample preparation process, unlike the nucleic acid isolation step, is varied and needs to be customized to the biological sample material (Huang et al., 2002, Anal Bioanal. Chem, 372:49-65, 2002).

Indeed, different types of tissue samples require different treatments before nucleic acid molecules can be extracted. The need for various treatments is a result of the inherent differences in the extracellular matrix compositions and inter-cellular connections in different tissues. For instance, muscle tissues and many cancer tissues are more fibrous and tougher in nature compared to brain or kidney tissues. These differences have led to the conventional method of mechanically disrupting and homogenizing solid tissues by manually using an electric hand-held device, typically a Dounce or Potter-Elvehjem “homogeniser”.

Despite increasing research on the automation for sample preparation in MEMs, much of the work has centred primarily on integrating simple cell lysis processes only. While many existing publications (e.g., U.S. Pat. No. 6,344,326) have presented integrated approaches for DNA separation starting from cells, integrated microfluidic, and/or MEM systems for nucleic acid isolation from solid tissue remain elusive and undemonstrated for two reasons: Firstly, cell samples are much easier to lyse and homogenise compared to tissue samples due to intercellular adhesions. Secondly, many standard methods for tissue homogenisation involve mechanical crushing and shearing forces, which are not MEMs friendly and pose significant obstacles to miniaturisation.

Such conventional manual and mechanical approaches to nucleic acid extraction have been standard bench top processes for many years. Multitudes of nucleic acid isolation kits are available commercially. Many are non-automated (e.g. Ambion, Amersham, Qiagen, TRizol kits, etc), providing only the chemical reagents and materials required for the nucleic acid isolation process. Some protocols like those of Dynal beads, incorporate automation into their isolation systems. However, these are at best semi-automated and still require a technician to perform many manual procedures and oversee the process. For instance, in many “automated” nucleic acid isolation kits, the homogenization process for the preparation of cell lysate from tissue is still performed manually with an electric homogeniser, one sample at a time, resulting in the need for frequent washes of the homogeniser tip to prevent cross contaminations. According to a first embodiment the invention provides a method for isolating nucleic acid molecules from tissue samples comprising:
i) treating a tissue sample with at least one enzyme for tissue dissociation;
ii) adding a lytic solution;
iii) isolating nucleic acid molecules and/or proteins.

The enzymolytic tissue dissociation allows a more efficient dissociation of the tissues than the conventional mechanical homogenization methods, as cells are less clustered together. Further, the enzymolytic tissue dissociation essentially maintains the cells intact such that the RNAases and proteases are not released from the cells. Hence, nucleic acid molecules are not destroyed and the nucleic acid molecules isolation can be carried out efficiently. Further, as the dissociation of the tissue sample is not performed manually and without using an electric homogeniser, the need for frequent washes of the homogeniser tip is avoided. This also prevents cross contaminations. Furthermore, as the homogenization step is avoided, the method of the invention is faster and less labour-intensive than the mechanical homogenization method.

The enzyme for tissue dissociation and the tissue sample are preferably incubated in solution at a controlled temperature, preferably 37° C. until tissue is softened almost completely and tissue dissociation visually appears to be complete.

According to another aspect, the method of the invention further comprises a step of applying hydrodynamic shear force to the product of step (i).

After enzymolytic tissue dissociation the softened tissue sample is then passed through a specially designed disruption channel to further fragmentize and release cells by the flow force generated by a pump or created by aspiration method (vacuum). Besides employing chemical enzymolysis to disrupt a tissue sample, the present method and system also utilizes hydrodynamic shear force to break up the tissue sample. In this way the resulting cells are efficiently released from the disrupted tissue sample. The cell yield of the tissue disruption process is high as the cells are substantially fully released from the tissue sample.

According to a further embodiment, the invention relates to a method for isolation of cells from tissue samples. The isolated cells can be stored and preserved and used for future applications. Alternatively, they may be subjected to further steps of lysis and isolation of nucleic acid molecules. In order to isolate the nucleic acid molecules, further steps of lysis and isolation are carried out as described below. The isolated cells can also be used for the preparation of proteins. Methods known in the art can also be used by the skilled person to isolate proteins during the dissociation and/or disruption steps.

Accordingly, the present invention provides a method for cell isolation from tissue samples comprising:
(a) treating a tissue sample with at least one enzyme for tissue dissociation;
(b) applying hydrodynamic shear force to the product of step (a);
(c) recovering the isolated cells.

The method further comprises adding a lytic solution to the isolated cells and recovering nucleic acid molecules.

For the purpose of the present invention, the term “tissue dissociation” means a tissue sample treated with at least one enzyme for dissociation, for example, at least a protease, cellulase, or lipase, or a mixture thereof. As a result of the tissue dissociation the tissue sample is softened and only a portion of the cells is released. The term “tissue disruption” refers to a tissue, which has been dissociated by using at least an enzyme for dissociation, further subjected to hydrodynamic shear force. After the tissue disruption step the cells are substantially fully released from the tissue sample.

Tissues suitable for use in the present invention are fresh tissues as well as preserved tissues, including frozen tissues treated with preservatives. Tissues can be animal-, human-, or agriculture-originated tissues. Tissue samples includes, for example, any kind of animal or human biological tissue sample, plant tissue or adipose tissue. Tissue source can include, without limitation, forensic, medical, agricultural, and research samples; tissues taken from different organs; tissues processed immediately or stored at liquid nitrogen or preservative reagents until analysis. Tissues processed immediately or stored until analysis; frozen, unfrozen, thawed, and never frozen tissues. The term tissue, as used herein, is an article that can be degraded by a tissue dissociation enzyme or by an enzymatic process. The tissues preferably contain at least two cells and a biomatrix. Extracellular matrices, polysaccharide matrices, and collagen are examples of a biomatrix. The weight of the tissue can range from 1 mg to 10 mg.

The size of the tissue sample is preferably between 1 to 10 mm³. The smaller sizes are preferable so that penetration of the sample by a tissue dissociation enzyme is facilitated. Tissue samples may be prepared, for example, by taking a biopsy of tissue with an appropriately sized biopsy tool, or a tissue may be cut into tissue samples to achieve the desired volume. Embodiments of the invention are suitable for use with preserved tissues, including frozen tissues and tissues treated with preservatives, for example the product RNAlater® (Ambion, Qiagen).

A further aspect of the present invention is that blood and/or body fluids may also be used in the method described. For example, when cells are to be isolated from blood and/or body fluid. Body fluid is a general term which refers to body fluids such as tears, sweat, urine, gastric and intestinal fluids, as well as saliva, various mucous discharges, and sinovial fluids. Blood and body fluids can be used in the method and system of the invention for the extraction of nucleic acid molecules.

The enzyme for tissue dissociation may be chosen according to the tissue sample used.

In particular, enzyme for tissue dissociation is a protease or a mixture thereof.

The protease may be collagenase, trypsin, chymotripsin, elastase, papain, chymopapain, hyaluronidase, pronase, dispase, thermolysin, bromelain, cathespines, or pepsin, or a mixture thereof. The most preferred protease is collagenase since it degrades collagen, which is a chief component of most tissues.

Combinations of proteases may also be used. Some proteases are very specific in action, and produce a limited cleaving action while others completely reduce a protein to individual amino acids. Accordingly, some proteases may be chosen if a particular tissue is known to be rich in a certain protein or biomolecule.

The enzyme for tissue dissociation may also be a cellulase when the tissue sample is a plant or plant-derived tissue. The enzyme for tissue dissociation may be a lipase when the tissue sample is an adipose or adipose-derived or associated tissue sample.

In case a combination of one or more of the above tissue sample is used, a mixture of at least two of the above enzyme for tissue dissociation can be used.

Other enzymes for tissue disruption suitable for the purpose of any embodiment of the present invention known in the art can also be used.

The tissue disruption is preferably performed so that the cells in the tissue remain intact. The cells may optionally be separated from the tissue debris, for example by a mechanical filtration step. The isolated cells can also be used for the preparation of proteins. Methods known in the art can also be used by the skilled person to isolate proteins during the dissociation and/or disruption steps.

The cells may optionally be sorted before lysis, for example by using a cell sorter that recognizes markers on the cells. The homogenized tissue product is optionally washed to remove proteases and is subjected to a cell disruption step, preferably performed by introducing the product into a lysis solution.

Conventional cell lysis techniques may be used to disrupt the intact cells. Table 2 describes some of these methods. Some of these methods may be used to preferentially recover one particular subcellular fraction. For example, conditions can be chosen such that only cytoplasmic fractions are released, or intact mitochondria or other organelles are recovered by differential centrifugation. Sometimes these techniques are combined, (e.g., osmotic lysis following enzymatic treatment, freeze-thaw in the presence of detergent). Proteases may be liberated when cells are lysed so that cell disruption is preferably performed at low temperatures. The sample may optionally be protected from proteolysis, and is preferable if the time between disruption and denaturation of cellular proteins is significant.

TABLE 2
Convetional cell lysis processes

| Cell disruption | | General |
|---|---|---|
| method | Application | Procedure |
| Osmotic lysis: | Blood cells, | Suspend cells in |
| Gentle method is | tissue culture | a hypoosmotic |
| well suited for | cells | solution. |
| applications in | | |
| which the lysate | | |
| is to be subsequent- | | |
| ly fractionated | | |
| into subcellular | | |
| components. | | |
| Free-thaw lysis: | Bacterial cells, | Rapidly freeze |
| Many types of cells | tissue culture | cell suspension |
| can be lysed by | cells | using liquid |
| subjecting them | | nitrogen then |
| to one or more | | thaw. Repeat if |
| cycles of | | necessary. |
| quick freezing and | | |
| subsequent thawing. | | |
| Detergent lysis: | Tissue culture | Suspend cells in |
| Detergents solubilize | cells | lysis solution |
| cellular membranes, | | containing deter- |
| lysing cells and | | gent. Cells can |
| liberating their | | often be lysed |
| contents. | | directloy into |
| | | sample solution |
| | | because these |
| | | solutions always |
| | | contain detergent. |
| Enzymatic lysis: | Plant tissue, | Treat cells with |
| Cells with cell walls | bacterial cells, | enzyme in |
| can be lysed | fungal cells | isoosmotic |
| gently following | | solution. |
| enzymatic removal | | |
| of the cell wall. | | |
| This must be done | | |
| with an enzyme | | |
| specific for the | | |
| type of cell | | |
| to be lysed | | |
| (e.g., lysozyme | | |
| for bacterial cells, | | |
| cellulase and | | |
| pectinase for plant | | |
| cells, lyticase for | | |
| yeast cells). | | |

Nucleic acid molecules and/or proteins can be isolated from the product of the lysis step according to any standard technique known in the art.

Matrixes, carriers, membrane filters, and the like may be conveniently used to adsorb, bind, retain or trap the nucleic acid molecules and/or proteins. The nucleic acid molecules and/or proteins are then recovered and isolated from the matrixes, carriers, membrane filters, and the like. Examples of carriers, matrixes and membrane filters include glass, silica gel, anion exchange resin, hydroxyapatite and celite such as Diatomaceus Earth. The shape of the matrixes, carriers, and membrane filter is not particularly limited. They can be in the form of beads, mesh filters or powder. For example, they may be in the form of glass filter, glass beads or glass powder.

According to one particular aspect, the nucleic acid molecules, which include mRNA, RNA and/or DNA, may be isolated by: adding beads coated with at least one linker and recovering the nucleic acid molecules bound to the linkers. or example, mRNA may be isolated by using beads coated with at least one inker comprising oligo d(T). The oligo d(T) recognizes and binds to the poly (A) of the mRNA.

According to another example, mRNA, RNA and/or DNA may be isolated by using at least one linker wherein the free end of the linker comprises at least one nucleotide N, wherein N is A, G, C, T or U. For example, linker comprising NNNN, NNNNN, NNNNNN can be conveniently used. This technique is known as the “universal linker” technique. An example of it is described in EP1325118 A (herein incorporated by reference). More in particular, the “universal linkers” are randomly generated.

Any method known in the art may be conveniently used to recover the beads, mesh filters or powder to which the nucleic acid molecules and/or proteins are bound to. Beads may be captured by using mechanical barrier. For example, by using a flow-through filter-chamber for bead trapping as described in Helene Andersson, 2001, “Microfluidic devices for biotechnology and organic chemical applications”, Royal Institute of Technology (KTH), Stockholm, Sweden (http://www.lib.kth.se/Sammanfattningar/andersson011116.pdf) (herein incorporated by reference). Another alternative method consists of selectively trapping non-magnetic beads in a monolayer in microfluidic devices (systems) without the use of physical barriers. This method involves microcontact printing and self-assembly, that can be applied to silicon, quartz or plastic substrates. In the first step, channels of the device are etched in the substrate. The surface chemistry of the internal walls of the channels is then modified by microcontact printing. The device is submerged in a bead solution and beads self-assemble based on surface chemistry and immobilize on the internal walls of the channels. (Helene Andersson, as above).

The beads may be magnetic beads coated with at least one linker. The nucleic acid molecules can be recovered by using an external magnetic field (external magnets) or magnets integrated into the device (system).

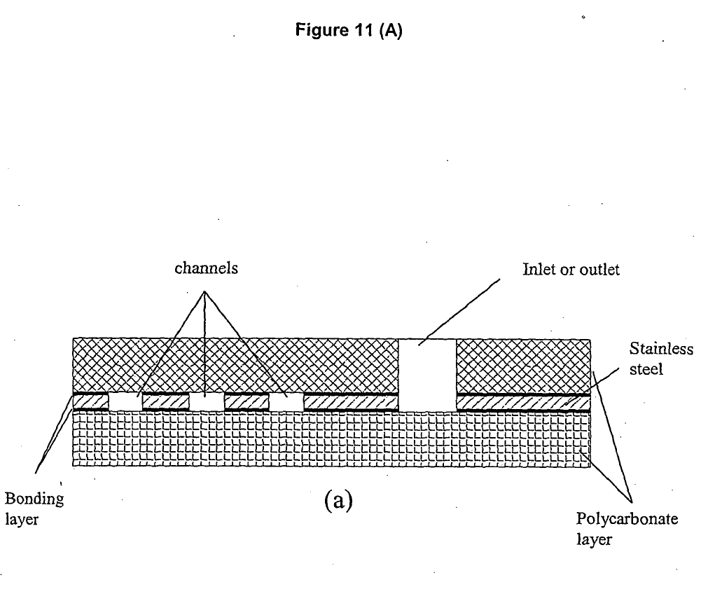

The present invention also provides a system for isolation of nucleic acid molecules from tissue samples, the system comprising an enzymolytic tissue dissociation chamber and a tissue disruption channel (see FIG. 10).

In particular, the system for isolation of nucleic acid molecules from tissue samples, comprises at least:
a first chamber for incubation of a mixture of: at least one tissue sample, at least one enzyme for dissociation of the tissue sample, and buffer solution;
a second chamber acting as a tissue disruption channel;
a third chamber including a lytic solution;
a fourth chamber for the collection and isolation of nucleic acid molecules and/or proteins; and
a fifth chamber for waste collection;
wherein the chambers are connected to each other.

The tissue disruption channel comprises:
an inlet port;
at least one region of constriction; and
an outlet port.

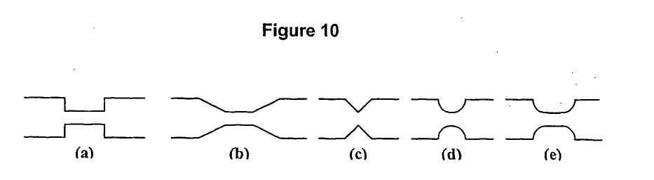

At the region(s) of constriction the cross-sectional area is smaller compared to the overall cross-sectional area of the disruption channel (see FIGS. 9 and 10).

The enzymolytic tissue dissociation chamber accepts at least one tissue sample and at least one enzyme for tissue dissociation. The type of tissue(s) and enzyme(s) are as described above.

The emzymolytic tissue dissociation chamber may be used as a micromechanical device, and therefore may be conveniently adapted to use with small tissue samples and small volumes of enzymes. The chamber is therefore preferably less than 100 μl in volume and the sample is preferably less than 10 mm³ in volume. Smaller volumes are more preferable.

Tissues suitable for use in the present invention are fresh tissues as well as preserved tissues, including frozen tissues treated with preservatives. Tissues can be animals and/or human-originated tissues. Tissue source can include, without limitation, forensic, medical, agricultural, and research samples; tissues taken from different organs; tissues processed immediately or stored at liquid nitrogen or preservative reagents until analysis. Tissues processed immediately or stored until analysis; frozen, unfrozen, thawed, and never frozen tissues. The term tissue, as used herein, is an article that can be degraded by a protease or an enzymatic process. The tissues preferably contain at least two cells and a biomatrix. Extracellular matrices, polysaccharide matrices, and collagen are examples of a biomatrix. The weight of the tissue can range from 1 mg to 10 mg.

Smaller sizes of the tissue are preferable so that penetration of the sample by a protease is facilitated. Tissue samples may be prepared for example, by taking a biopsy of tissue with an appropriately sized biopsy tool, or a tissue may be cut into tissue samples to achieve the desired volume. As said above, plant tissues or adipose tissues may also be used in any embodiment of the invention. Embodiments of the invention are suitable for use with preserved tissues, including frozen tissues and tissues treated with preservatives, for example the product RNAlater® (Ambion, Qiagen).

A further aspect of the present invention is that blood and/or body fluids may also be used in the method and system of the invention. For example, blood and/or body fluids can be place into a system (device) according to any embodiment of the invention and cells can be isolated from blood and/or body fluids using the hydrodynamic shear forces. Further, nucleic acid molecules can be extracted from the cells isolated from blood and/or body fluids.

The enzyme for tissue dissociation may be chosen according to the tissue sample used.

In particular, enzyme for tissue dissociation is a protease or a mixture thereof.

The protease may be collagenase, trypsin, chymotripsin, elastase, papain, chymopapain, hyaluronidase, pronase, dispase, thermolysin, bromelain, cathespines, or pepsin, or a mixture thereof. The most preferred protease is collagenase since it degrades collagen, which is a chief component of most tissues.

Combinations of proteases may also be used. Some proteases are very specific in action, and produce a limited cleaving action while others completely reduce a protein to individual amino acids. Accordingly, some proteases may be chosen if a particular tissue is known to be rich in a certain protein or biomolecule.

The enzyme for tissue dissociation may also be a cellulase when the tissue sample is a plant or plant-derived tissue. The enzyme for tissue dissociation may be a lipase when the tissue sample is an adipose or adipose-derived or associated tissue sample.

In case a combination of one or more of the above tissue sample is used, a mixture of at least two of the above enzyme for tissue dissociation can be used.

Other enzymes for tissue disruption suitable for the purpose of any embodiment of the present invention known in the art can also be used.

The system according to the invention is preferably a biological microelectromechanical system (bioMEMS) and/or a fully automated complete micrototal analytical system (μTAS).

The system of the invention further comprises a chamber containing matrixes, carriers, membrane filters, and the like in order to conveniently adsorb, bind, retain or trap the nucleic acid molecules. The nucleic acid molecules are then recovered and isolated from the matrixes, carriers, membrane filters, and the like. Examples of carriers, matrixes and membrane filters include glass, silica gel, anion exchange resin, hydroxyapatite and celite such as Diatomaceus Earth. The shape of the matrixes, carriers, and membrane filter is not particularly limited. They can be in the form of beads, mesh filters or powder.

The chamber may comprise of a mechanical barrier to capture beads, which have nucleic acid molecules bound to them. For example, the chamber may include flow-through filter-chamber for bead trapping as described in Helene Andersson, 2001, as above.

In particular, beads coated with at least one linker for isolation of nucleic acid molecules may be used. For example, the beads are magnetic beads and are recovered by using an external magnetic field. Alternatively, magnets may be integrated into the system.

A further embodiment of the invention is that the system may be an automated nucleic acid extractor. For example, the different chambers of the system may be linked such that there is minimal need for human intervention, thus leaving less room for contamination, errors and possibly cutting down on the overall process time. The system can also be a disposable automated system for tissue sample preparation. For example, a nucleic acid extractor for the purposes of genomic or proteomic analyses.

Furthermore, the present invention provides a method for isolating nucleic acid molecules using the system as described above.

The invention also provides a method for cells from a tissue sample, the method comprising at least:
incubating in a first chamber a mixture of: at least one tissue sample, at least one enzyme for dissociation of the tissue sample, and buffer solution;
disrupting the tissue sample in a second chamber acting as tissue disruption channel;
optionally a chamber for cells collection, and
optionally a chamber for waste collection;
   the chambers being optionally connected to each other.

In particular, the invention provides a method for isolating nucleic acid molecules from a tissue sample, the method comprising at least:
incubating in a first chamber a mixture of: at least one tissue sample, at least one enzyme for dissociation of the tissue sample, and buffer solution;
disrupting the tissue sample in a second chamber acting as tissue disruption channel;
lysing cells isolated from the tissue disruption channel in a third chamber; and
collecting and isolating desired nucleic acid molecules in a fourth chamber;
   the chambers being optionally connected to each other.

Any one of the system (device) of the invention optionally comprise a port for input tissue sample, and inlet and outlet of the tissue disruption channel for connecting fluids and pump, respectively.

The incubation in the first chamber may be carried out at a suitable temperature. For example, the incubation can be carried out at a constant temperature, preferably 37° C. The incubation time is interdependent of the size of the tissue sample. Suitable incubation duration evident by a skilled person in the art is chosen. A shorter time will have poor yield of RNA while a longer incubation will time will result in the degradation of RNA.

The hydrodynamic shear force applied within the tissue disruption channel gradually reduces the tissue sample size until it is fully disrupted and cells are released.

The nucleic acid molecules, which include mRNA, RNA and/or DNA, are collected from the solution according to any standard method known in the art. For example, by adding beads coated with at least one linker and recovering the nucleic acid molecules bound to the linkers. The beads may be magnetic beads and collected by an external magnetic field or by magnets integrated into the system.

For example, mRNA may be isolated by using beads coated with at least one linker comprising oligo d(T). The oligo d(T) recognizes and binds to the poly d(A) of the mRNA.

According to another example, mRNA, RNA and/or DNA may be isolated by using at least one linker wherein the free end of the linker comprises at least one nucleotide N, wherein N is A, G, C, T or U. For example, linker comprising NNNN, NNNNN, NNNNNN can be conveniently used. This technique is known as the “universal linker” technique. An example of it is described in EP1325118 A (herein incorporated by reference). More in particular, the “universal linkers” are randomly generated.

Certain embodiments of the invention may greatly simplify and improve the tissue dissociation and disruption processes. They help to overcome many obstacles in bioMEMs in the process of sample preparation, and enable accelerated development of complete μ-TAS, which are capable of performing nucleic acid molecule and/or protein isolation from tissue samples, for example from solid tissue sample, in a completely automated fashion. An embodiment of the invention is a method wherein a clinician deposits a clinical sample in a receptacle and the entire nucleic acid molecule and/or protein isolation process takes place without further human intervention. The purified nucleic acid molecules are collected in a chip and stored appropriately until required for further use.

Certain embodiments of the invention include articles, devices or systems preferably MEMS, bioMEMS and/or μTAS that include an enzymolytic tissue dissociation chamber. An enzymolytic tissue dissociation chamber refers to a chamber that accepts at least one tissue sample and at least one enzyme but does not accept or use a device for mechanically homogenizing the tissue. Thus an enzymolytic tissue dissociation chamber does not function with a mechanically acting device that homogenizes tissue, for example a grinder. Also, the enzymolytic tissue dissociation chamber dissociates a tissue by accepting at least one tissue sample and at least an enzyme, preferably one of the proteases disclosed herein, an equivalent thereof, or a mixture thereof. The enzymolytic tissue dissociation chamber is preferably adaptable as a MEMS, bioMEMS and/or μTAS device and therefore is preferably adapted to use with small tissue samples and small volumes of enzymes. The chamber is preferably less than 100 μl in volume and the sample is preferably less than 100 μl in volume. Smaller volumes are more preferable, with less than 50 μl in volume being more preferable, less than 10 μl volume being yet more preferable, and less than 5 μl volume being most preferable.

The enzymolytic tissue dissociation chamber is preferably operably associated with other chambers. The other chambers have other functions involved in tissue dissociation and/or disruption, cell disruption, or nucleic acid molecules processing, isolating and/or analysis. The other chambers may include, without limitation: chambers for proteases or other enzymes for tissue dissociation, protease inhibitors, buffers, washes, detergents, chemicals, solutions, salts, or reagents; waste collection points; inlet ports; outlet ports; product collection chambers; and analysis chambers. For example, the inlet and outlet of the tissue disruption chamber is for connecting to fluid input and a pump respectively.

Separation processes may also be operably associated with the chambers described herein. For example, a filter may be used to separate dissociation and/or disruption products by size. Other separation processes may also be performed.

Certain embodiments of the invention are a MEMS, bioMEMS and/or μTAS device that incorporates on-chip sample preparation, including tissue dissociation using enzymatic methods and tissue disruption according to any embodiment of the invention. The MEMS may be single monolithic devices or several microfluidic modules, which are associated with or integrable with each other. The bioMEMS device may include processes of PCR amplification, electrophoresis, expression profile microarray analysis, genotyping, etc. Alternatively, the MEMS can be incorporated into an integrated micro-analytical system to perform the downstream amplification and detection functions after nucleic acid isolation which can be applied to diagnostics, drug discovery or biomedical research. Examples of MEMS or bioMEMS that perform some of these functions are found in U.S. Pat. Nos. 6,675,817; 6,468,800; 6,468,761; 6,447,661; 6,440,725; 6,387,710; 6,375,817; 6,238,922; 6,221,677; 6,179,595; 5,952,215; 5,786,207; 5,667,985; 5,443,791; 5,374,395, which are hereby incorporated by reference herein.

Another embodiment of this invention is using this method in MEMS, bioMEMS and/or μTAS based micro fluidic device or system for automatic bio-sample preparation. In this embodiment, a method that employs both chemical enzyme and hydrodynamic shear force for fresh tissue and frozen tissue dissociation is provided.

The process for bio-sample preparation comprises incubating at least one tissue sample in an incubation chamber, optionally, with buffer comprising at least one enzyme for dissociation of the tissue, for example, protease (e.g. Trypsin, collagenases, or the like), cellulae, or lipase, or a mixture thereof. Temperature and time are controlled until the tissue sample is softened. Cells are released partially from the tissue sample in this step. As the digestion procedure is controllable, digestion reaction can be terminated by the time that each individual cell is released from the tissue sample. In this stage, any species of bio-molecule, especially RNAs, are well protected by the intact cell compartments. In the intact and viable cells, RNase, which is the main protease to destroy the bio-molecules, is well kept essentially within the lysosomes.

The softened tissue sample is then passed through a specially designed disruption micro-channel to further fragmentize and release the cells by the flow force generated by a pump or created under aspiration (under vacuum). Besides employing chemical enzymolysis to dissociate the tissue sample, the present device also utilizes hydrodynamic shear force to break up the tissue sample so that it becomes small enough to pass through the disruption channel.

The tissue disruption channel consists of tissue disrupting components. Each tissue-disrupting component consists of an inlet port (orifice), region(s) of constriction and an outlet port (orifice). The region(s) of constriction has a smaller-cross sectional area as compared to the inlet/outlet port. With a constant liquid flow-rate through the disruption channel, the flow velocity is much greater at the region(s) of constriction than at the inlet or outlet ports. The softened tissue stretches along the direction of flow and squeezes through disruption components. This softened tissue is thus cruxed (crunched) into small pieces by the shear force generated by the rapid velocity profile (ripple). Tissue fragmentation takes place as the tissue passes through the tissue disruption components.

The isolated cells from the tissue sample are then subjected to a cell lysis step. The cell lysis step is performed by introducing the mixture into a channel and mixing it with lysis buffer. The lysate is subjected to nucleic acid molecule. For example, poly (A)+ RNA isolation through magnetic beads, which is also compatible with MEMS, bioMEMS and/or μTAS. Total messenger RNAs are obtained in purified form, and are suitable for the detection of specific gene expressions.

The advantages of the invention include MEMS, bioMEMS and/or μTAS and microfluidic compatibility, high efficiency, absence of cross-contamination, reduction in the required sample size, automation and possibility of high output and so on.

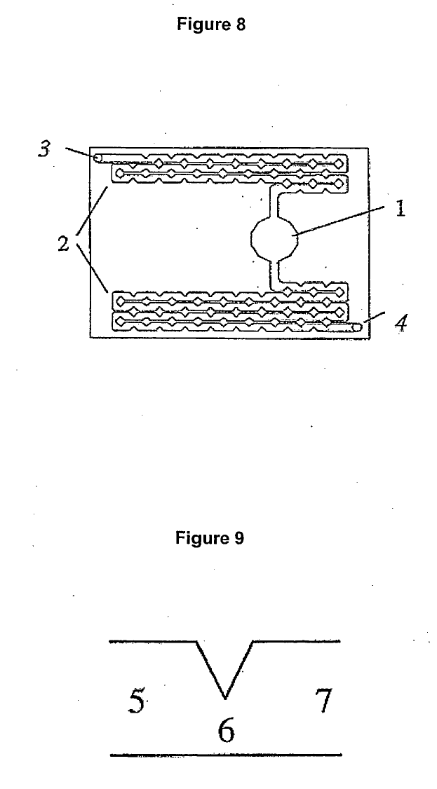

The invented microfluidic tissue disruption device comprises at least a sample incubation chamber, a series of tissue disruption channels, an inlet and an outlet. A micropump or syringe pump can be connected externally or integrated inside the device. Alternatively, the fluid movement can be created by applying aspiration methods. One example of the system according to the invention is shown in FIG. 8.

An important feature of the system of the invention is the tissue disruption channel. It is constructed by a series of tissue disrupting components. Each tissue-disrupting component comprises at least an inlet port, a region(s) of constriction and an outlet port as shown in FIGS. 9 and 10. The region(s) of constriction has a sharp edge. The ratios of the inlet port/outlet port to the region(s) of constriction vary from 2-5 along the channel. The size of the orifice also changes along the channel to avoid tissue from being stuck in the disruption components. This design also increases the disruption efficiency. Some possible designs of the disruption component are shown in FIG. 10.

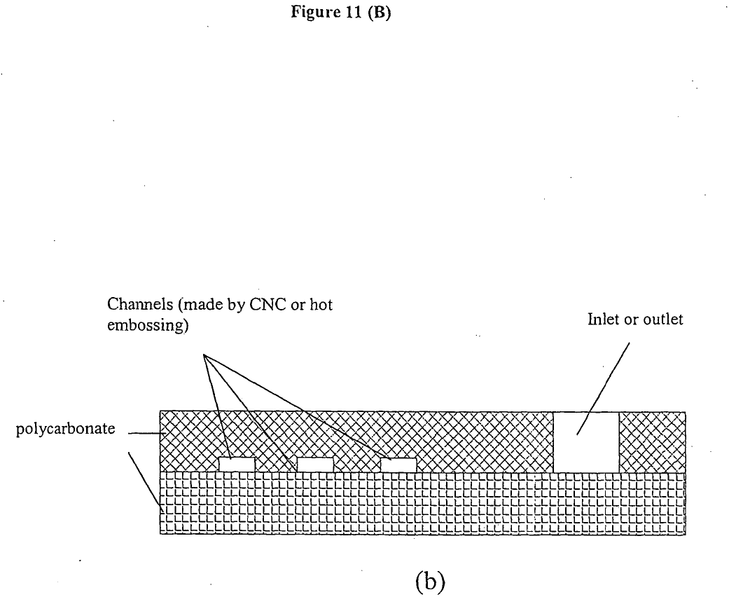

An example of this device, which has a sandwich structure, is shown in FIG. 11A and 11B. The lower layer and upper layer of the device are made of polycarbonate using CNC milling machine. The middle layer consists of almost all the features of the disruption device. This layer is fabricated in thin stainless steel plate with 200-1000 um-thickness using laser cutting machine. The upper layer, lower layer and middle layer are bonded together by a bonding layer (VST Acrylic Foam Tape).

The design of this particular example is to demonstrate the working principle of the invention. This, however, shall not limit the usage of other designs and dimensions.

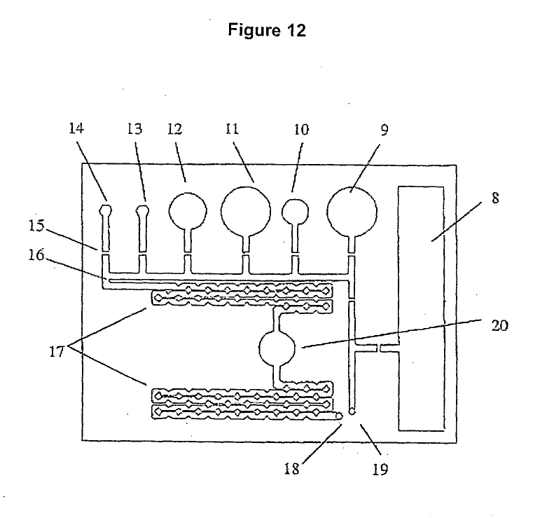

The fabrication methods for such a device can also make use of other methods like etching in micro-machining, molding and hot embossing. FIG. 12 is an example using the invented technology for disruption tissue sample; subsequently, the extraction and purification of the biomolecules are required.

The system of the invention may be made of any suitable material. For example, glass, silicon or plastic may be used. Plastic and polymers such as polystyrene, polycarbonate and poly-methyl-methacrylate provide a cheaper and disposable system.

A further embodiment of the invention is that the system may be used as part of a diagnostic integrated system suitable for forensic testing, clinical diagnostics, veterinary and/or agricultural diagnostics.

Having now generally described the invention, the same will be more readibly understood through reference to the following examples which are provided by way of illustration, and are not, intended to be limiting of the present invention.

### EXAMPLES

### Example 1

Trypsins and collagenases were used as exemplary models of certain embodiments of the invention. The process set forth herein are however applicable to other types of tissues, including human tissues, plant tissues, adipose tissues, and the like.

Trypsin-EDTA digestion of rat liver was carried out as follows: freshly harvested tissue was cut into 2 mm³ sample sizes, followed by washing twice in 500 μl iced Phosphate Buffered Saline (PBS). Trypsin-EDTA solution was added to the tissue sample, which was incubated in a shaking water bath at 37° C. for 30 min, and triturated from time to time until no further tissue disruption was observed. A similar procedure was followed using collagenase, except that: 1) incubation time was increased to 90 min and shaking was not necessary; and 2) gentle flicking of the sample was applied instead of trituration after incubation. The cell suspension obtained using these procedures yielded a homogenous solution that could be used for downstream RNA isolation by TRIzol directly without pelleting or washing the cells.

A series of experimental parameters were studied, including sample treatment, enzyme selection, enzyme concentration and volume, digestion duration and application of physical agitation. Cell viability counting was carried out as a direct monitoring of the digestion performance. RNA isolation from the cell suspension by TRIzol was conducted to examine the influence of enzymatic digestion in RNA preservation. RNA yield and purity were checked by UV-visible spectroscopy. RNA integrity was checked by agarose gel electrophoresis.

For sample treatment, incubation of sample in trypsin-EDTA at 4° C. overnight before digestion was found to be comparable to the other methods conventionally used for tissue dissociation. It was also found that 2 mm³ size of tissue, which is approximately the size of a biopsy sample, was effectively digested. Further dissection made no significant difference in the digestion performance. As for enzyme selection, trypsin-EDTA, collagenase type I, IV and VIII were all proven to be effective in isolating cells.

As for enzyme concentration and volume, 0.01% to 0.25% of trypsin-EDTA was effective, while 0.01% to 0.15% was found to be preferable. Other concentrations could be used, however, by adjusting the time of exposure to the protease. Generally, a higher enzyme volume in a range of 20 μl to 500 μl afforded higher cell yields. Cell yield from using 20 μl of trypsin enzyme was about 40% of the yield from using 500 μl enzyme. For collagenase, 500 μl of 200 U/ml enzyme solutions were used for tissue digestion. As for the digestion time, for trypsin-EDTA digestion, 30 min was found to be effective. For collagenase digestion, 1 to 2 hours was effective. Table 3 shows further experimental conditions.

TABLE 3
Experimental settings of tissue digestion by enzyme.

| Enzyme type | Volume | | Concentration | Reaction time | Agitation |
|---|---|---|---|---|---|
| Trypsin-EDTA | 500 | μl | 0.05% | 30 min | Shaking, |
| | | | | | Pipetting |
| Collagenase | 500 | μl | 200 U/ml | 90 min | Flicking |
| type I | | | | | |
| Collagenase | 500 | μ1 | 200 U/ml | 90 min | Flicking |
| type IV | | | | | |
| Collagenase | 500 | μl | 200 U/ml | 90 min | Flicking |
| type VIII | | | | | |

The number of cells isolated from 10 mg (2 mm³) rat liver tissue is about 10⁶ cells per mg of tissue. Cell viability evaluated by trypan blue was found to be between 97% to 100%.

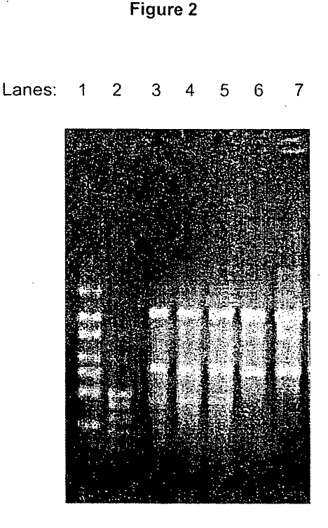

RNA isolated from the enzyme digestion approach was compared with that from a conventional homogenization approach. Gel electrophoresis images of total RNA are shown in FIG. 2: Agarose gel of total RNA run in TBE. Lane from left to right: Lane 1: high range RNA marker 6 kb, 4 kb, 3 kb, 2 kb, 1.5 kb, 1 kb, 0.5 kb; Lane 2: low range RNA marker 1 kb, 0.8 kb, 0.6 kb, 0.3 kb; Lane 3: total RNA isolated by collagenase type I; Lane 4: total RNA isolated by collagenase type IV; Lane 5: total RNA isolated by collagenase type VIII; Lane 6: total RNA isolated by trypsin-EDTA; Lane 7: total RNA isolated by homogenization. The presence of the two distinctive rRNA bands at 28 S and 18 S indicates that the total RNA species were well-preserved.

In general, the approach afforded similar results to conventional processes, such as that reported (Chomczynski, P., 1993, Biotechniques 15, 532) using homogenization (60-100 mg; Invitrogen Protocol). An OD ratio of A260 to A280 was found to be 2.08 to 2.12 measured in PH 7.4 PBS buffer, which indicates the RNA was of high purity.

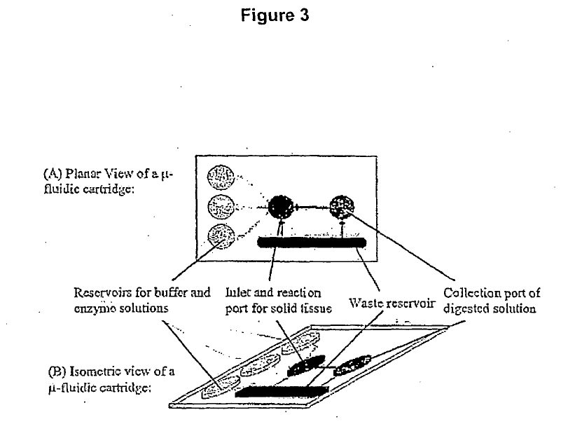

One possible scheme for implementing enzymatic tissue digestion in a bioMEM system is shown in FIG. 3, which depicts the design of a μ-fluidic car fridge consisting of (1) chambers for buffer and protease solutions; (2) inlet and reaction ports for a solid tissue sample; (3) a collection, port for the digested solution; and (4) a waste chamber. In addition, the illustrated μ-fluidic cartridge could also be integrated with other downstream bioMEMs processes, such as cell lysing, nucleic acid separation and detection. Another example is as seen in FIG. 8, which consists of (1) chambers for buffer and protease solutions (not shown in the figure); (2) inlet and incubation chamber 1 for a solid tissue sample; (3) channel 2 for disruption of the softened tissue; (4) inlet 3 for connecting the buffer and protease solutions; (5) micro-pump or syringe pump connection port 4.

According to alternative embodiments, the device of the invention can be made in a wide range of materials typically used for microfabricated systems. These include, but are not limited to, materials such as a silicon wafer, silica wafer, polydimethylol siloxane (PDMS), polycarbonate and polymethyl methacrylate (PMMA).

### EXAMPLE 2

Trypsin and collagenases were used as exemplary models of this embodiment. As an example, Trypsin-EDTA digestion of rat liver was carried out as follows: freshly harvested tissue was cut into 8 mm³ (10 mg in weight) sample size, followed by washing twice in 500 μl iced Phosphate Buffered Saline (PBS). Trypsin-EDTA solution was added to the tissue sample, which was incubated in a shaking water bath at 37° C. for 30 min, pipette the solution until no further tissue disruption was observed. A similar procedure was followed using collagenase except that: 1) incubation time was increased to 90 min and shaking force was not necessary; and 2) gentle flicking was applied instead of triturating after incubation.

By our experiment, 0.01% to 0.15% of trypsin concentration was found to be preferable in terms of cell yield. Other concentrations could be used, however, by adjusting the time of exposure to the protease. Table 4 is the optimized experimental trypsin concentration for fresh tissue and frozen tissue. For fresh rat liver tissue, cell yield was about 1×10⁵ cells/mg.

TABLE 4
Experimental settings of tissue digestion by enzyme

| | Enzyme | | | Concen- | Reaction | | |
|---|---|---|---|---|---|---|---|
| Sample | type | Volume | | tration | time | | Agitation |
| Fresh | Trypsin- | 500 | μl | 0.05% | 30 | min | Shaking, |
| | EDTA | | | | | | pipetting |
| Frozen | Trypsin- | 500 | μl | 0.1% | 2 | min | Shaking, |
| | EDTA | | | | | | pipetting |

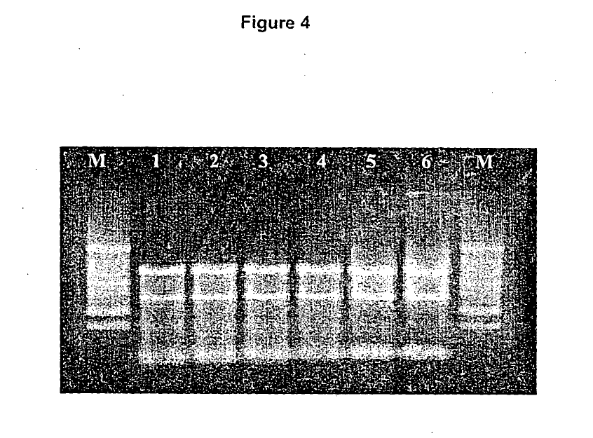

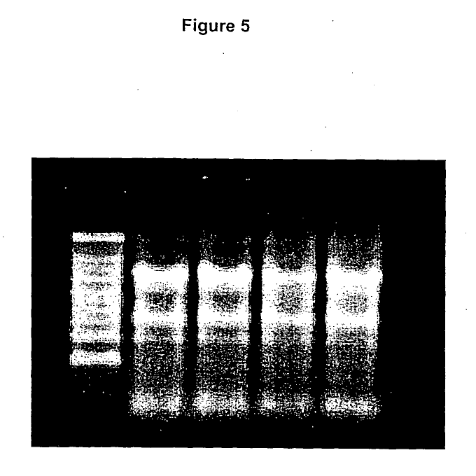

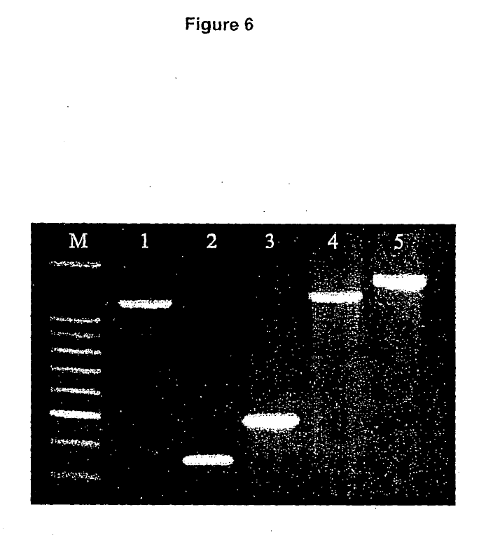

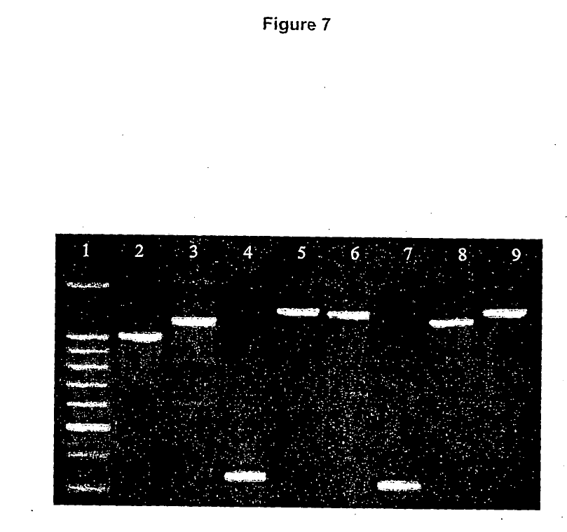

The cell suspension obtained using these procedures yielded a homogeneous solution that could be used for downstream RNA isolation by TRIzol or magnetic beads. The RNA yield was 50-100 μg from 10 mg rat liver tissue, which was comparable to that reported (Chomczynski, P., 1993, Biotechniques 15, 532) using homogenisation (60-100 μg, Invitrogen protocol). An OD ratio of A260 to A280 was found to be 2.08 to 2.12 measured in pH 7.4 PBS buffer, which indicates the RNAs were of high purity. Total RNAs from fresh and frozen tissues using the invented dissociation method are not degraded in term of intactness of ribosomal RNAs shown in FIG. 4 and 5, respectively. Table 5 shows the total RNA yield comparison. The data shows that the total RNA yield variation is small. Several selected full-length genes, like 13 actin, (3-microglobulin, cyclophilin, TP53 and c-myc can be amplified from rat liver tissues with high quality (FIG. 6). Instead of mRNA isolation from rat liver tissues, the human breast tissue from fibrosarcoma patients have been examined using several specific markers for breast tumor. The specific breast tumor markers like CD59, keratin 19, TP53, Histone H4 Maspin as well as α-antichymotrypsin can be detected shown in FIG. 7. It indicates that our method has effectively isolated RNAs from animal tissues as well as cultivated cell lines. This invention is compatible with automation of MEMS device and is highly useful for screening/differentiating gene expression among various tissues that are normal, benign or malignant in molecular diagnosis.

TABLE 5
Total RNA comparison

| | | | Yield |
|---|---|---|---|
| Method | Sample | A₂₆₀/A₂₈₀ | (μg/10 mg) |
| Trypsin | T1 | 2.04 | 48.18 |
| digestion | T2 | 2.04 | 56.89 |
| | T3 | 2.02 | 56.54 |
| | T4 | 2.02 | 52.62 |
| | Mean | 2.03 | 53.56 |
| Homogenizer | H1 | 2.02 | 69.93 |
| | H2 | 1.85 | 88.85 |
| | Mean | 1.94 | 79.39 |

### EXAMPLE 3

The process of the tissue disruption device including the following steps:

100 μl of protease [0.05-0.15% (wt/vol) for Trypsin and 100-300 unit/ml for collagenases] solution is first injected into the incubation chamber and preheated to 37° C. Fresh or frozen mammal tissue (up to 10 mg) is then put into the chamber and sealed. The tissue sample is incubated inside the chamber for about 15 minutes so that it becomes softened by the enzymolysis of the protease solution.

Once the incubation time is over, the softened tissue and the solution are passed through the disruption channel for tissue disruption with the help of a micropump, which is connected, to the inlet and outlet of the device (Refer to FIG. 12, Components 18 & 19). Shear force generated in the disruption components breaks the softened tissue into smaller size. These smaller pieces of tissue will, then be softened with the enzymolysis of the protease reagent. As the dimension of the disruption components becomes smaller, the tissue size becomes reduced gradually until it is fully disrupted and cells are released. The total tissue dissociation time (incubation time and disruption in micro channel time) is about 25 minutes.

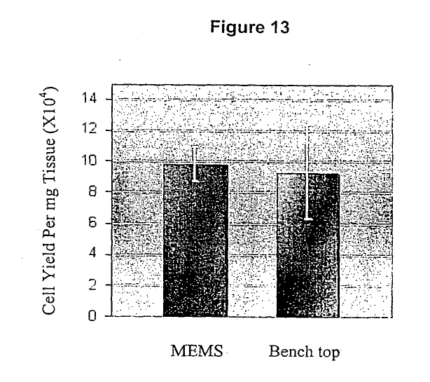

For fresh rat liver, the average cell yield is 9.85×10⁴ cells per mg tissue sample. The cell yield is slightly higher than the standard lab method, which uses motorised mechanical homogeniser and protease for tissue disruption. The average cell yield for the standard lab method is 9.35×10⁴. FIG. 13 shows the comparison between the two methods as mentioned.

The cells obtained from the disrupted tissue sample are then passed through the lysis step for extraction of DNA, RNA and mRNA depending on the requirement.

In this particular example, mRNA is extracted. As shown in FIG. 12, the disrupted cells are passed through micro disruption/mixing channel with lysis/bonding buffer to break down the cells. After 15 minutes, the cell membrane is fully broken up. DNA, RNA mRNA, protein and other intracellular components are dissolved in the solution. Magnetic beads (from Dynalbeads or Bionobile magnetic beads) with poly d(T) oligos are passed through the mixing channel to capture mRNA inside the solution and then these beads are collected by an external magnetic filed. Debris is removed using 4 washing steps inside the mixing channel. The mRNA is purified after the washing steps. Finally, elute reagent is passed through the mixing channel to separate the mRNA from the magnetic beads.

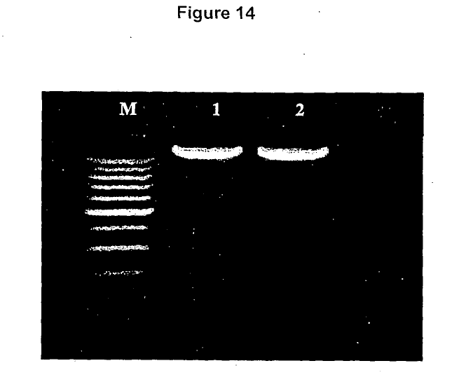

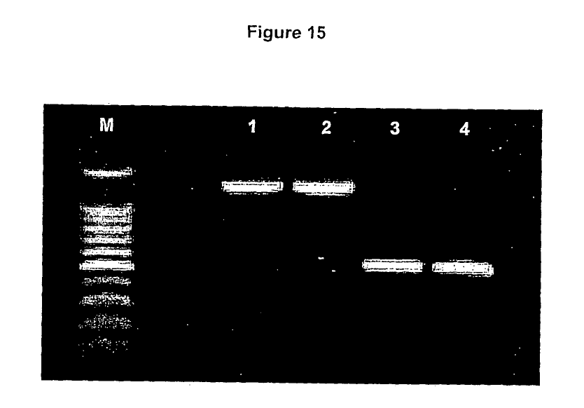

The mRNA extracted from microfluidic device is amplified by a RT-PCR step outside the device. FIG. 14 shows the gel electrophoresis for the synthesis of Bata-actin mRNA extracted from 3 mg of fresh rat liver tissue. FIG. 13 shows the gel electrophoresis for the synthesis of TP53 and cyclophilin mRNA from the above mentioned sample. We can conclude that the gene is intact.

For the synthesis of TP53, the yields from using microfluidic device and using motorized homogeniser was 2730 ng and 2920 ng, respectively. For the synthesis of cyclophilin, the yield from using microfluidic device and using motorised homogeniser was 2270 ng and 2280 ng respectively.

We can conclude that the yield from using the microfluidic device is as high as the conventional method that gives the highest yield. The total process time for extraction and purification of mRNA by the microfluidic device will take less than 45 minutes.

The patents, patent applications, and publications set forth in this application (including the appendices of the application) are hereby incorporated by reference herein. The embodiments of the invention set forth herein are merely exemplary and are not intended to limit the scope of the invention.

1. A method for isolating nucleic acid molecules from tissue samples comprising:
i) treating a tissue sample with at least one enzyme for tissue dissociation;
ii) adding a lytic solution;
iii) isolating nucleic acid molecules.

2. The method of claim 1, further comprising a step of applying hydrodynamic shear force to the product of step (i).

3. The method of claim 2, the method comprising:
incubating in a first chamber a mixture of: at least one tissue sample, at least one enzyme for dissociation of the tissue sample, and buffer solution;
disrupting the tissue sample in a second chamber acting as tissue disruption channel;
lysing cells isolated from the tissue disruption channel in a third chamber; and
collecting and isolating desired nucleic acid molecules and/or proteins in a fourth chamber.

4. The method of claim 3, wherein the incubation in the first chamber is carried out at a constant temperature.

5. The method of claim 3, wherein hydrodynamic shear force applied within the tissue disruption channel gradually reduces the tissue sample size until it is fully disrupted and cells are released.

6. (canceled)

7. The method of claim 1, wherein the enzyme for tissue dissociation is a protease, cellulase and/or lipase.

8. (canceled)

9. (canceled)

10. (canceled)

11. (canceled)

12. (canceled)

13. (canceled)

14. (canceled)

15. A device for isolation of cells and/or nucleic acid molecules from tissue samples, the device comprising an enzymolytic tissue dissociation chamber and a tissue disruption channel.

16. (canceled)

17. The device of claim 15, comprising:
a first enzymolitic tissue dissociation chamber for incubation of a mixture of: at least one tissue sample, at least one enzyme for dissociation of the tissue sample, and buffer solution; and
a second chamber acting as a tissue disruption channel.

18. The device of claim 15, further comprising a chamber for recovery of the isolated cells.

19. The device of claim 15, comprising:
a first enzymolitic tissue dissociation chamber for incubation of a mixture of: at least one tissue sample, at least one enzyme for dissociation of the tissue sample, and buffer solution;
a second chamber acting as a tissue disruption channel;
a third chamber comprising a lytic solution;
a fourth chamber for the collection and isolation of nucleic acid molecules and/or proteins; and
a fifth chamber for waste collection;
wherein the chambers are connected to each other.

20. The device of claim 15, wherein the tissue disruption channel comprises:
an inlet port;
at least one region of constriction; and
an outlet port.

21. The device of claim 20, wherein the tissue disruption channel at the region(s) of constriction has a smaller cross-sectional area compared to the overall cross-sectional area of the disruption channel.

22. The device of claim 15, wherein the enzymolytic tissue dissociation chamber accepts at least one tissue sample and at least one enzyme for tissue dissociation.

23. The device of claim 15, wherein the enzymolytic tissue dissociation chamber is less than 100 μl in volume.

24. (canceled)

25. (canceled)

26. (canceled)

27. The device of claim 17, wherein the enzyme for tissue dissociation is a protease, a cellulase or a lipase.

28. (canceled)

29. (canceled)

30. (canceled)

31. The device of claim 15, wherein the device is a biological microelectromechanical system (bioMEMS) and/or a fully automated complete micrototal analytical system (μTAS).

32. The device of claim 15, wherein the device is disposable.

33. (canceled)

34. (canceled)

35. A method for cell isolation from tissue samples comprising:
(a) treating a tissue sample with at least one enzyme for tissue dissociation;
(b) applying hydrodynamic shear force to the product of step (a);
(c) recovering the isolated cells.

36. The method of claim 35, further comprising: adding a lytic solution to the isolated cells.

37. The method of claim 35, further comprising:
recovering nucleic acid molecules.

38. (canceled)

39. The method of claim 35, wherein the enzyme for tissue dissociation is a protease, cellulase and/or lipase.

40. (canceled)

41. (canceled)

42. (canceled)

43. (canceled)

44. (canceled)

45. (canceled)

46. (canceled)
